(19) United States
(12) Patent Application Publication
(10) Pub. No.: US 2004/0071793 A1
(43) Pub. Date: Apr. 15, 2004

(54) Oral rehydration methods and compositions

(76) Inventor: Paul J. Bobrowski, Scottsdale, AZ (US)

Correspondence Address: ELLIS & VENABLE, PC, 101 NORTH FIRST AVE., SUITE 1875, PHOENIX, AZ, 85003, US

(21) Appl. No.: 10/678,817

(22) Filed: Oct. 3, 2003

Related U.S. Application Data
(60) Provisional application No. 60/416,714, filed on Oct. 5, 2002.

(51) Int. Cl. A61K 33/14; A61K 35/78; A61K 31/70

(52) U.S. Cl. 424/725; 514/23; 424/680; 424/717; 514/53

(57) ABSTRACT

An improved oral rehydration solution comprising standard formulae of sugars, mineral salts and bicarbonates in combination with Croton species materials with or without the inclusion of material from the Uncaria species. The botanical components are concentrated biologically active materials and thus enhance therapeutic benefit. The Croton component functions as an effective non-paralytic agent in managing diarrhea that is superior to loperamide resulting in a lower stool output. The functionality of the Uncaria species is as an effective anti-inflammatory agent via NF-□B activation inhibition and protecting gastrointestinal epithelial cells from oxidant-induced death.

This application claims benefit of U.S. Provisional Application serial No. 60/416,714 filed on Oct. 5, 2002.

### BACKGROUND OF THE INVENTION

Field of the Invention

In mammals, there are a variety of factors which may cause gastrointestinal distress accompanied by a loss of fluid (e.g. vomiting, diarrhea). If transient, the condition may go untreated. However, often the event can cause a fluid loss accompanied by dehydration, such as in some cases of pediatric or amoebic diarrhea, necessitating intervention.

Diarrhea is primarily addressed by either the administration of an agent which absorbs pathogenic bacteria, digestive enzymes, toxins, and nutrients from the gastrointestinal tract, or the administration of an oral electrolyte/sugar replacement fluid, more commonly as an oral rehydration solution (ORS). In the latter approach the goal is to simply replace fluid and electrolytes that have been lost, in order to avoid dehydration and electrolyte imbalance.

An electrolyte, sugar, bicarbonate solution has been the standard course of ORS therapy for the treatment of diarrhea throughout the world with the primary focus being the replacement and maintenance of lost fluids. Safe and effective values of these components are well established. The gold standard as recommended by the World Health Organization (WHO) contains 20 grams glucose, 90 milliequivalents (mEq) sodium, 30 mEq bicarbonate and 20 mEq potassium per liter water.

While the WHO formula is effective, it does not prevent fluid/nutrient loss but acts to replace at a rate greater than that of depletion. A means of replacing critical fluids and nutrients while at the same time preventing or reducing the active rate of fluid and electrolyte loss would represent a significant improvement over current ORS therapy with widespread application in both human and animal health.

Ethnomedical treatments for human disease are primarily derived from native botanicals. In the case of the indigenous Amazonians, latex (sap) from the bark of Croton species as well as decoctions from the bark of the Uncaria species have both been used in the treatment of gastrointestinal distress. However, while effective, the traditional ethnomedicines have undesirable properties which limit their use.

Sangre de grado, the viscous latex derived from various Croton species plants found primarily in the Amazon River basin, is an effective agent in managing diarrhea. It is not a paralytic like loperamide but rather it works through the same mechanism as for its analgesic properties. Sensory afferent nerves drive secretory responses in the gut and preparations of sangre de grado have been shown to block epithelial secretion in response to sensory afferent nerve activation. Acute fluid shifts in response to gut injury induced by acid and undigested protein (a model of bacterially driven intestinal necrosis) are blocked by preparations of sangre de grado at dilutions of 1:1000 (200 μg/ml). In addition to preventing the secretory response, it reduces the associated damage to the intestinal mucosa. Neurally mediated epithelial secretion is characteristic of various diarrheal events, and sangre de grado is an effective blocker of epithelial secretion of electrolytes as determined by sensory afferent nerves.

### SUMMARY OF THE INVENTION

Aspects of the invention are summarized below to aid in the understanding of embodiment(s) of the invention and the application. Yet, the invention is fully defined by the claims of the application.

An electrolyte, sugar, bicarbonate solution has been the standard course of ORS therapy for the treatment of diarrhea throughout the world with the primary focus being the replacement and maintenance of lost fluids. However, this current treatment addresses the symptoms of the event but not the cause.

The present invention generally comprises methods and compositions for oral rehydration in humans and animals that simultaneously reduces fluid loss. The methods and compositions disclosed herein contain botanical derivatives that retain the ability to inhibit emesis and activation of sensory afferent nerves, thus reducing causative agents (i.e. vomiting, diarrhea) in combination with fluids and nutrients that address symptoms through rehydration. The methods and compositions disclosed herein incorporate botanical derivatives from the Uncaria and Croton species that retain their medicinal benefits with an electrolyte/sugar composition containing a potassium salt, a sodium salt, bicarbonate and a sugar. The invention herein relates more particularly to the combination of the prior art anti-diarrheal compositions (i.e. ORS) together with the novel botanical Uncaria and Croton preparations and extracts. The purpose of these combinations with botanicals is to attenuate the processes that promote fluid and electrolyte loss while at the same time replacing what has already been lost. It provides for a treatment that addresses both the symptoms and cause of the condition and thus offers novel benefit over current therapies by reducing the severity and duration of the conditions.

Emesis often accompanies enteric infections, resulting in a variety of manifestations of cause for concern (i.e. dehydration, loss of medication, etc). In a well-established in vivo model, a novel lipid extract of the Croton specie sangre de grado (Zangrado) blocked emesis at concentrations of 3 mg/kg. Thus, extracts and preparations of Sangre de grado are effective anti-diarrheal agents that arrest emesis and offers substantial mucosal protective properties. Cat's Claw of the Uncaria species, is an effective antioxidant and it has long been known that a sign of inflammation is an increased production of oxidants and free radicals. Oxidants promote diarrhea by epithelial electrolyte secretion as well as by promoting damaging gut epithelia, thereby promote a ‘“leaky’ gut barrier secondary to epithelial cell death and dysfunction. A novel preparations or extracts of the Uncaria specie (Vincaria) prevents cell death in response to the toxic nitrogen oxide and peroxynitrite, oxidants (i.e. H₂O₂) and free radicals, implicated in gut inflammation, cell death and epithelial secretory response.

According to one aspect of the invention, compositions are incorporated into biologically active dosage units forming oral rehydration anti-diarrheal compositions. The final composition may be transported in a dehydrated, powdered state, in a liquid state, in a confectioned state (i.e. candy, jello, pudding, etc.) or as a food additive. If in a powdered state, some measure can be taken to prevent moisture from causing solidification of said powder, which would interfere with fluid reconstitution (i.e. moisture-resistant packaging and/or a desiccating agent and/or crystallization). Additionally, the final compositions may also contain coloring, flavoring and sweetening agents for better palatability.

### BRIEF DESCRIPTION OF THE FIGURES

FIG. 1. The extraction process significantly (“*”) reduces the proanthocyanidin content of the parent latex (SdG). When combined in a base vehicle, such as Aloe barbadensis shown here, the extract (CGO 110) produced a mixture absent of the intense color seen in similar preparations with the parent latex. This change, which is readily quantifiable by spectrophotometer, negates the discolorizing (i.e. staining) properties commonly associated with proanthocyanidins and the parent latex and allows for practical dermatological preparations;

FIG. 2. A prototypical activator of sensory afferent nerves, capsaicin, was topically applied to the mucosal surface of the stomach in anesthetized rats and mucosal blood flow measured by a Laser Doppler Flow meter. The marked increase in mucosal blood flow induced by 300 μM capsaicin was largely prevented by either the parent material, Sangre de grado or its extract, Zangrado (CGO 110) at doses of 2 and 0.2 mg/ml, respectively, indicating that the organic extract retains the ability to effectively prevent the activation of sensory afferent nerves;

FIG. 3. Changes in short circuit current (measured as lsc) in guinea pig ileal mucosal preparations housed in Ussing chambers can demonstrate that sangre de grado inhibits epithelial secretion of electrolytes, as indicated by prevention of a change in current, induced by sensory afferent nerves but not the neuropeptide Substance P. Capsaicin, the prototypical activator of these sensory afferent nerves promotes a change is short circuit current, and this is readily blocked by sangre de grado, whereas direct activation of the epithelial cells by Substance P remains unaffected. This clearly establishes that sangre de grado can negate neurogenic epithelial secretion.

FIG. 4. Using a well-established ferret model of post-operative complications of nausea, emesis and itch induced by morphine, the organic extract of sangre de grado, Zangrado (CGO 110) was administered intraperitoneally (3 mg/kg) to ferrets 15 minutes prior to the subcutaneous injection of 0.05 mg/kg of morphine-6-glucuronide (M6G). Administration of M6G caused a significant number of vomiting (2.2±0.4) and retching (10±1.2) incidences in the control group while in those animals pre-treated with Zangrado, the number of these episodes was virtually abolished (vomiting 0.6±0.3; retching 2.2±0.7, P<0.05). It is clear that this organic extraction procedure contains active components and is effective in the treatment of emesis.

FIG. 5. A comparison of Uncaria parent and extract by high performance liquid chromatography (HPLC). As shown in the overlaid chromatograms, the Uncaria extract derived from the methods described herein (Vincaria™) is substantially deplete of the immunostimulating alkaloids found in the parent (Uncaria spp) whilst retaining and thus enhancing the efficacy and therapeutic potential of the polar, immunosuppressive and TNF-alpha inhibiting (anti-inflammatory) components.

FIG. 6. Oxidants and free radicals can readily promote the death of epithelial cells. Here three oxidants that are structurally distinct, a free radical (DPPH), and two oxidants (peroxynitrite and hydrogen peroxide) promote death of gastric epithelial cells in culture. Inclusion of cat's claw in the form of the alkaloid-deplete Vincaria extract, significantly reduced cell oxidant-induced cell death, as evidence for the ability of Vincaria to maintain the integrity of the epithelial barrier.

### DESCRIPTION OF EMBODIMENTS

Extraction Procedures

According to one aspect of this invention, an organic extraction of Croton species latex concentrates its lipophilic components and reduces the hydrophilic proanthocyanidin content of the plant material. The invention demonstrates that extract CGO 110 (Zangrado) reduces vomiting and diarrhea and is selectively cytotoxic to cancerous cells, unlike the parent material, representing an improvement in safety and therapeutic activity.

According to one aspect of this invention, plant material or an aqueous extract of plant materials from the Uncaria species are subjected to organic solvents which concentrates its polar biologically active components while reducing its lipophilic components. The invention demonstrates that the extract is effective at a dose twenty times less than extracts or decoctions produced by other methods and much less than the parent material, representing a significant improvement in therapeutic activity and potentials.

According to one aspect of this invention, the extraction and removal of water through physical manipulation concentrates the biologically active components in both the Croton species latex and Uncaria species aqueous extract. The invention teaches that these extracts are more therapeutically effective at lower concentrations than the parent material, representing a significant improvement in therapeutic potential and use.

Preferred methods to accomplish the aforementioned species Uncaria and Croton extractions have been previously described but it is contemplated that a skilled practitioner could devise variations of the procedures given the disclosure herein and the desired results.

Extraction Process 1 for Croton Species.

Latex, or sap from Croton species is mixed with an organic solvent. The preferred organic solvent is ethyl acetate although other organic solvents can be used as would be obvious to the ordinarily skilled practitioner in light of the disclosure herein. In other embodiments, the preferred organic solvent is isopropanol, a chloroform/Methanol mixture, or an equivalent thereof. The organic solvent is added to the latex in a 1:1 proportion. In the preferred extraction process the solvent latex combination is agitated.

The preferred agitation method is stirring although other agitation methods are also contemplated to be effective. Following agitation, the mixture is settled, or allowed to settle into distinct phases including at least an organic layer and an aqueous layer. The organic phase or layer is comprised largely of solute lipophilic materials, representing the active constituent, and a significantly reduced quantity of proanthocyanidin components relative to the pre-agitation step. The organic layer is separated from the aqueous layer for further processing pursuant to the preferred extraction process.

Moreover, it is common to find a gel-like substance in the organic layer at the interface of the aqueous and organic layers. This gel substance is characterized as having a dark brown and purple color and comprises hydrophilic constituents trapped with water. In the preferred process the gel substance is processed further to separate any active lipophilic constituents from the hydrophilic constituents. The preferred manner of processing the gel substance is the addition of a drying agent to the organic layer or the gel substance. The preferred drying agent is magnesium sulfate in a concentration of 0.5-5 g/L of contaminant gel. It is contemplated that other equivalent drying agents at relative effective concentrations would also be effective and would be obvious to the ordinarily skilled practitioner in light of the disclosure herein and with undue experimentation.

The addition of the drying agent results in a precipitant, which traps water and hydrophilic constituents or water-based colored chemical contaminants. The precipitant can be readily separated from the hydrophilic constituents by filtration or other techniques known to separate precipitants. Actual laboratory procedures achieved acceptable results using a Whatman #4 filter paper or an equivalent.

The steps of organic extraction, mixing with a drying agent and filtration may be repeated up to three times to accomplish a thorough extraction of the active lipophilic constituents. At this point in the process, the lipophilic materials are solutes contained within the organic solvent, which are concentrated by evaporation of the solvent by one of several procedures, such as vacuum drying, freeze drying or heating. Actual heating up to 60 degrees Celsius produced acceptable drying results.

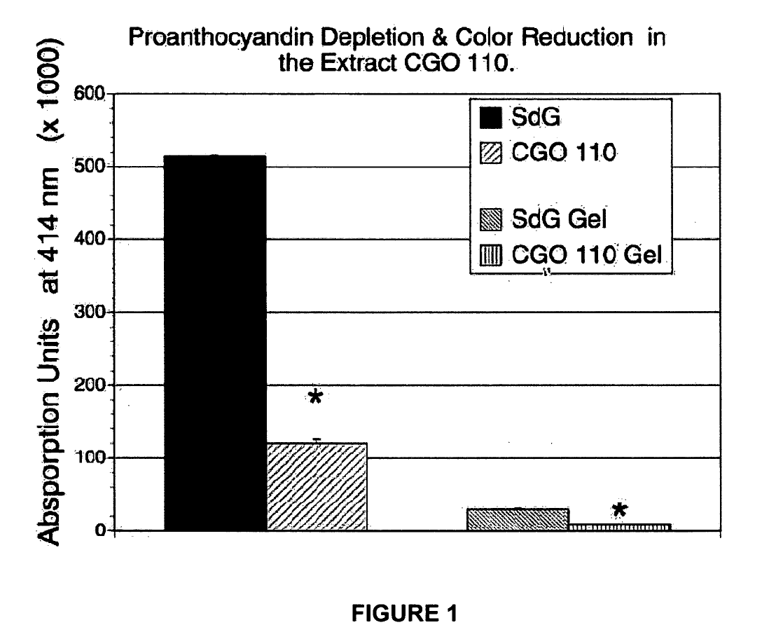

The organic layer composition thus processed is rich in lipophilic materials but largely clear of hydrophilic contaminants. Following the extraction process, the color of the organic layer can be characterized as a rose. Moreover, the reduced proanthocyanidin content is quantifiable spectrophotometrically. Relative absorbance of the extraction in the visible spectrum was compared to the absorbency peak of the parent latex (414 nm) in the visible range. At a concentration of 1 mg of extracted latex to 1 mL of water the disclosed process yielding the extraction (CGO 110) results in a 4.3 fold reduction in absorbance at 414 nm, as indicated in FIG. 1. This assessment was repeated 9 times with similar results achieved (significance difference P<0.0001, as denoted by the “*”). Similarly when sangre de grado or the extraction (CGO 110) at a concentrations of 200 μg per mL of aloe vera gel were applied to aloe vera gel to mimic their administration as topical products, there was also a significantly lower color response with the extracted sangre de grado, CGO 110 vs. the parent botanical (* P<0.0001). See FIG. 1. Estimates from the absorbency measurements indicate that the proanthocyanidin content was reduced by at least 90% relative to the nonextracted parent latex.

Extraction Process 2 for the Croton Species.

The latex from the Croton species is dried to its residual solid matter by methods such as heating, air-drying, vacuum or freeze-drying. The dried latex is rich in proanthocyanidin compounds and therefore characterized by a dark burgundy color. To the dried latex matter the organic solvent, ethyl acetate or an equivalent, is added. The dried latex and organic solvent mixture is agitated and the organic solvent is removed for further processing according to the procedure described in Example 1. This process may be repeated up to three times to accomplish a thorough extraction all lipophilic materials in the organic layer and solvent. If any water bearing contaminants are present, the addition of drying agent followed by filtration as noted above, will remove these contaminants. Removing the ethyl acetate through various methods including heating, air-drying, vacuum or freeze-drying then isolates the solutes contained within this organic extract.

The extraction thus processed according to the disclosed processes is characterized by a significant reduction of proanthocyanidin compounds. The reduction of the proanthocyanidin compounds leaves the extraction significantly diminished in color producing compounds and yet amenable to health care applications.

Extraction Process 1 for the Uncaria Species.

An aqueous extract of the Uncaria species is preceded prior art decoction methods. A preferred decoction comprises a quantity of raw or dried botanical in hot water. More specifically, solid matter of the plant material such as roots, bark and or powders of genus Uncaria are mixed in such a ratio with water that when heated for a period of time at a temperature of approximately 90-100 degrees centigrade, with or without agitation, to yield a brown aqueous extract also known as a decoction or tea. Then, according to the invention, the decoction or tea is then filtered of all solid matter for further processing. The filtrate is subsequently dried to remove all aqueous components. Acceptable drying methods include air-drying, evaporation, or vacuum drying or an equivalent.

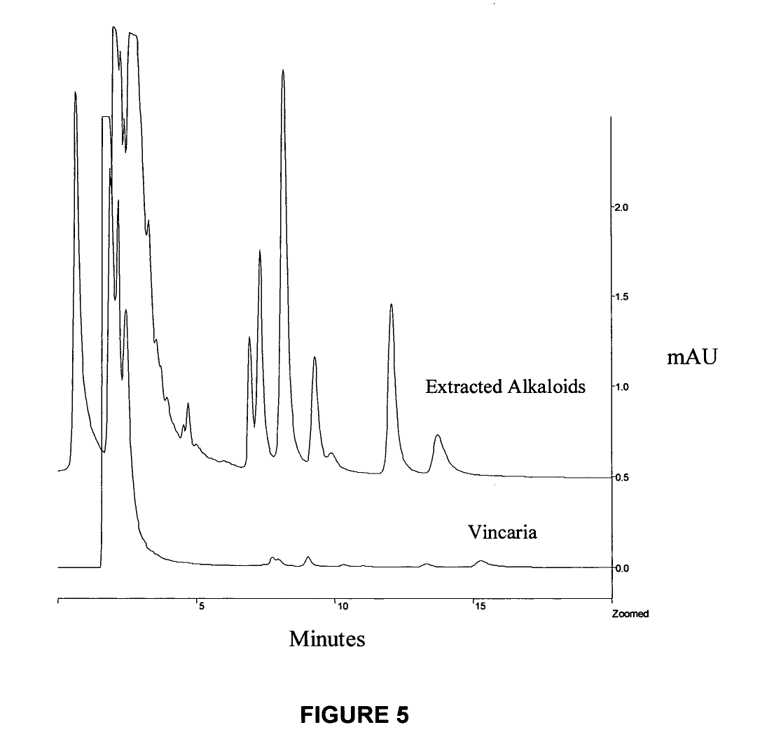

An organic solvent is subsequently added to the dried decoction or extract. A preferred organic solvent is chloroform/methanol (2:1) added in a volume-to-volume ratio of about 1:1 to 1:20. Another suitable organic solvent is ethyl acetate. Following mixing and phase settling the organic layer is removed from the aqueous layer for further processing according to one aspect of the invention. The solutes contained within the aqueous extract are then resolved by one of several drying processes: heating, air drying, freeze-drying or vacuum drying. Depletion of alkaloids content in the Uncaria extract is confirmed by high performance liquid chromatography (HPLC) as indicated in FIG. 5.

Extraction Process 2 for the Uncaria Species.

An aqueous extract of the Uncaria species is achieved by a decoction method as previously described. To this extract in its liquid phase the organic solvent ethyl acetate, is added. Following agitation and settling the organic layer is separated from the aqueous and the organic layer discarded. The solutes remaining in the aqueous layer are the resolved by drying. Several drying processes can be used: heating, air-drying, freeze-drying, or vacuum drying. Depletion of alkaloids content in the Uncaria extract is confirmed by high performance liquid chromatography (HPLC) as indicated in FIG. 5.

Extraction Process 3 for the Uncaria Species.

This process employs the same decoction extraction method as described for Examples 1 and 2, but before the extraction of organic material is employed; the solute in the decoction is isolated by drying. To this dried powder the organic solvents are added (chloroform/methanol, 2:1: or ethyl acetate). This mixture is agitated for adequate mixing, followed by settling. The liquid organic solvent is removed and discarded, and the solutes are dried again as outlined by similar methods employed for drying in Example 1 or 2. Depletion of alkaloids content in the Uncaria extract is confirmed by high performance liquid chromatography (HPLC) as indicated in FIG. 5.

Compositions

According to one aspect of this invention, the mixture of sugars, minerals and fluids in a fixed ratio and commonly known as an oral rehydration solution (ORS) can be combined with Croton species extracts (Zangrado) herein described. The invention demonstrates that the ORS-Zangrado combination (ZORS) is much more effective at treating gastrointestinal events characterized by a loss of fluid than current ORS therapy, Zangrado, other Croton extracts or the parent material alone. According to another aspect of the invention, the mixture of sugars, minerals and fluids in a fixed ratio (ORS) can be combined with Croton species extracts (Zangrado) and Uncaria species extracts (Vincaria) herein described. The invention demonstrates that the ORS-Zangrado-Vincaria combination (V-ZORS) is more effective at treating gastrointestinal events characterized by a loss of fluid than either current ORS therapies, Zangrado or other Croton extracts, Vincaria or other Uncaria extracts or the parent materials alone.

Effects of Zangrado on Sensory Afferents

Sensory afferent nerves mediate the sensations of pain, itch, cough and nausea and when activated by capsaicin, lead to a multitude of responses including vasodilation, inflammation, edema, and pain and itching. Using a well-established rat model, the mucosal surface of the stomach in anesthetized animals was inoculated with 300 μM capsaicin and mucosal blood flow measured by a laser Doppler flow meter.

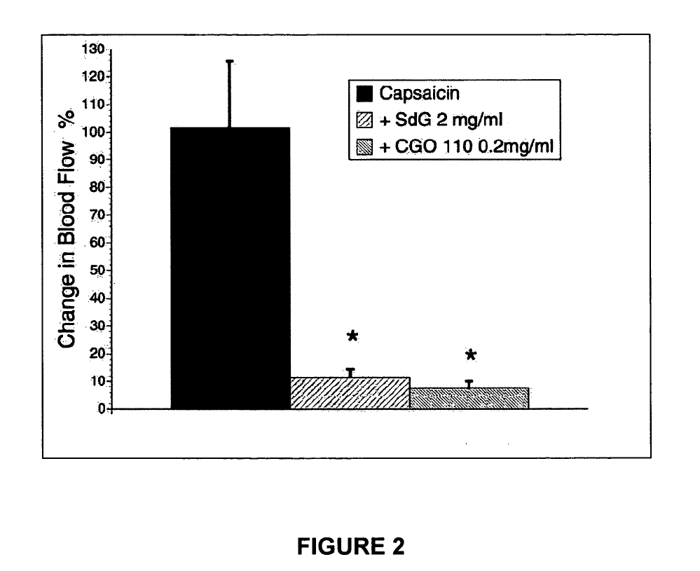

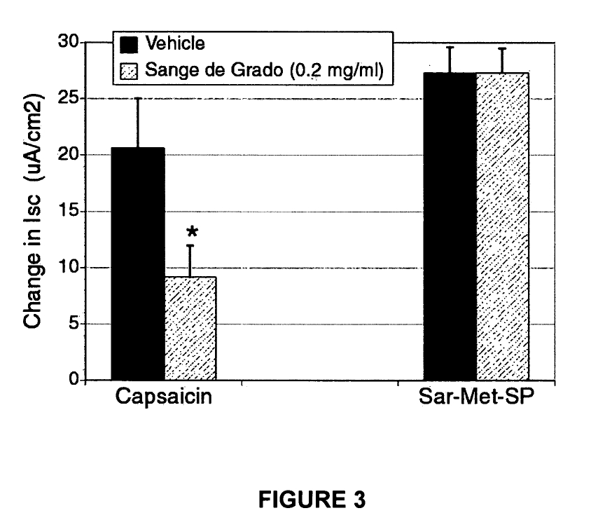

As indicated in FIG. 2, the marked increase in capsaicin-induced mucosal blood flow was prevented by either the sangre de grado parent material (SdG) or its organic extract Zangrado (CGO 110) at doses of 2 and 0.2 mg/ml, respectively. Thus, the sangre de grado organic extract described in this application retains the ability to effectively prevent the activation of sensory afferent nerves and offers significant benefit over the parent material. Evidence that Sangre de grado affects neurally-mediated electrolyte secretion is depicted in FIG. 3. Changes in capsaicin-induced short circuit current are attenuated by Sangre de grado, demonstrating that the locus of action is neurally mediated secretory events.

Effects of Zangrado on Morphine-Induced Emesis and Itch

Using a well-established ferret model of post-operative complications of nausea, emesis and itch induced by morphine, the extraction Zangrado (CGO 110) was administered intraperitoneally (3 mg/kg) to ferrets 15 minutes prior to the administration of morphine-6-glucuronide (M6G), known to promote itching, retching and vomiting. The animals were monitored for sixty minutes.

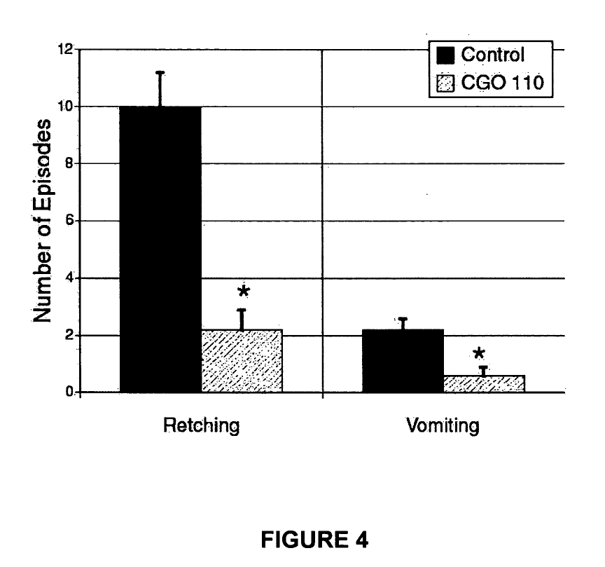

As shown in FIG. 4, the M6G caused a significant number of vomiting (2.2±0.4) and retching (10±1.2) incidences in the control group while in those animals treated with extract Zangrado, the number of episodes was virtually abolished (vomiting 0.6±0.3; retching 2.2±0.7, P<0.05). Itch as indicated by licking responses was reduced from a control value of 16.9±2.3 episodes to 2.2±0.7 in Zangrado treated animals (P<0.05). Thus, the extract offers significant improvement over the parent material or other preparations in the treatment of nausea and emesis.

Inhibition of TNFα Formation

The extract of Uncaria species processed according to the invention produced a composition with an enhanced ability to inhibit the formation of tumor necrosis factor alpha (TNFα) by macrophages stimulated by bacterial endotoxin [lipopolysaccharide (LPS)]. This activity is superior to that of other preparations or the parent material and therefore offers significant benefit in the treatment of gastrointestinal distress.

Table 1: Antioxidant and anti-TNFα actions of cat's claw formulations. Note that in order to assess activity in micropulverized Uncaria, a hot water extraction was performed and this decoction used for evaluation. TNFα production was assessed in cultured macrophages (RAW 264.7 cells) stimulated with bacterial endotoxin (LPS: 0.5 μg/mL). After one hour exposure to LPS, media was collected and TNFα levels measured by ELISA. DPPH scavenging was spectophometerically measured by a reduction in absorbance at 515 nm. Results are depicted as IC₅₀, which is the concentration that produces a 50% inhibition. A lower IC₅₀ value is indicative of greater potency. Note: For each assay, potency was significantly greater in alkaloid depleted freeze-dried formulation when compared to the other formations (P<0.01)

TABLE 1

| | Freeze-dried | | |
|---|---|---|---|
| | Alkaloid Deplete | Freeze-dried | Micropulverized |
| | Uncaria | Uncaria Alkaloid | Uncaria |
| Assay IC₅₀ | (Vincaria ™) | Intact | Alkaloid Intact |
| DPPH | 12.6 μg/mL | 20.8 μg/mL | 150 μg/mL |
| Anti-TNFα | 9.5 ng/mL | 14.1 ng/mL | 28 ng/mL |

Prevention of Oxidant-Mediated Epithelial Cell Death

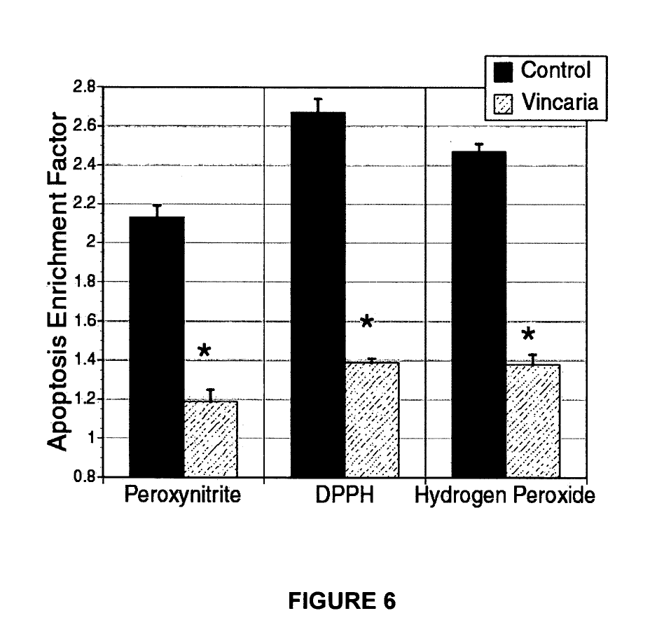

The extract of Uncaria species was found to be a very effective inhibitor of epithelial cell death induced by structurally divergent oxidants and free radicals (FIG. 6). Epithelial cell death leads to fluid and electrolyte loss as a consequence of a loss of the integrity of the barrier function. Inclusion of cytoprotective components in a standard ORS solution represents an improvement in the approach to reduce the duration and severity of the diarrheal symptoms.

Although the invention has been described in detail with reference to one or more particular preferred embodiments, persons possessing ordinary skill in the art to which this invention pertains will appreciate that various modifications and enhancements may be made without departing from the spirit and scope of the claims that follow.

What is claimed is:

1. A method of treating the symptoms of diarrhea in a mammals comprising administering a pharmacologically effective dose of a potassium salt, a sodium salt, a bicarbonate, a sugar, and botanical component selected from the group consisting of; an extract of from the plant material of the Croton species and an aextract of the plant material from the Uncaria species.

2. The method in claim 1 wherein the botanical component is a preparation of the latex of the Croton species containing less than about 10% water.

3. The method in claim 1 wherein the botanical component is an extract of the Croton species in a final concentration of about 20 to 300 micrograms per milliliter.

4. The method in claim 1 wherein the sodium salt is a chloride in a final concentration of about 1.0 to 5.0 milligrams per milliliter

5. The method in claim 1 wherein the potassium salt is a chloride in a final concentration of about 0.5 to 3.0 milligrams per milliliter.

6. The method in claim 1 wherein the bicarbonate is a potassium bicarbonate in final concentration of about 1.0 to 5.0 milligrams per milliliter.

7. The method in claim 1 wherein the sugar is selected from the group consisting of; sucrose and dextrose, in final concentrations of about 5.0 to 30.0 milligrams per milliliter.

8. The method in claim 1 wherein the botanical component is selected from the group consisting of an extract from the Uncaria species, in a final concentration of about 10 to 400 micrograms per milliliter.

9. The method in claim 1 wherein the pharmacologically effective dose further comprises an agent selected from the group consisting of, a suspending agent, a coloring agent, a flavoring agent, and a sweetening agent.

10. The method in claim 1 wherein the pharmacologically effective dose comprises about 20 to 300 milligrams of extract from the Croton species, about 10 to 30 grams of the sugar, about 60 to 120 milliequivalents sodium, about 10 to 30 milliequivalents potassium and about 10 to 50 milliequivalents bicarbonate per liter fluid.

11. The method in claim 1 wherein the pharmacologically effective dose is adminsterable in a form selected from the group consisting of, a liquid, a liquid concentrate, a liquid gelcap, a tablet, an effervescent, a capsule, a powder, a dietary supplement, a food, or a food additive.

12. The method in claim 1 wherein the pharmacologically effective dose includes a combination of Croton species material, sodium, potassium, bicarbonate and sugar which is effective in ameliorating diarrhea and oral rehydration.

13. The method in claim 1 wherein the pharmacologically effective dose includes Croton species extract with a selective cytotoxicity to cancerous cells.

14. The method in claim 1 wherein the pharmacologically effective dose includes Croton species material that ameliorates emesis and diarrhea.

15. The method in claim 1 wherein the pharmacologically effective dose includes Croton species material that inhibits the activation of sensory afferent nerves.

16. The method in claim 1 wherein the pharmacologically effective dose includes Uncaria species material that inhibits TNFα formation and inflammation.

17. The method in claim 1 wherein the pharmacologically effective dose comprises of botanical materials and nutrients which reduces emesis, diarrhea and fluid loss while replacing nutrients and fluids.
